(19) United States
(12) Patent Application Publication
(10) Pub. No.: US 2025/0143796 A1
(43) Pub. Date: May 8, 2025

(54) DEVICES, SYSTEMS, AND METHODS FOR JOINT PARAMETER VISUALIZATION

(71) Applicant: Mako Surgical Corporation, Weston, FL (US)

(72) Inventors: Matthew James CARTER, Auckland (NZ); Christina Esposito PARKER, Sydney (AU)

(73) Assignee: Mako Surgical Corporation, Weston, FL (US)

(21) Appl. No.: 18/934,415

(22) Filed: Nov. 1, 2024

Related U.S. Application Data
(60) Provisional application No. 63/596,019, filed on Nov. 3, 2023.

(51) Int. Cl. A61B 34/10; A61B 34/00; A61B 34/30

(52) U.S. Cl. CPC A61B 34/10; A61B 34/25; A61B 34/30; A61B 2034/105; A61B 2034/107

(57) ABSTRACT

Systems and methods are disclosed for receiving imaging data of a joint, determining gap parameter information based on the imaging data, generating a graphical user interface (GUI) configured to display a three-dimensional element comprising a plurality of sub-elements, determining which of the plurality of sub-elements correspond to the determined gap parameter information, and generating a display of the GUI, wherein at least one sub-element is associated with the determined gap parameter information.

### CROSS-REFERENCE TO RELATED APPLICATION(S)

This application claims the benefit of priority to U.S. Provisional Patent Application No. 63/596,019, filed Nov. 3, 2023, the entirety of which is incorporated by reference herein.

### FIELD OF THE DISCLOSURE

This disclosure relates to systems, devices, and methods for optimizing medical procedures, and in particular to systems, devices, and methods for joint parameter visualization based on ligament laxity and other parameters to facilitate joint balancing and optimize outcomes after joint replacement procedures, among other aspects.

### BACKGROUND OF THE DISCLOSURE

Surgeries incorporating prosthetics and/or implants, such as joint replacement procedures, often require careful consideration of various factors. An important factor for implant planning in, for example, a knee replacement is soft tissue balance assessment. The standard gap quadrants used for soft tissue balance assessment in total knee replacements include four sectors: medial extension, lateral extension, medial flexion, and lateral flexion. Current systems, methods, and devices for processing patient image data and ligament laxity information are difficult to visualize in relation to a patient's joint, time consuming for a physician to understand, and difficult to effectively use in a procedure that includes joint balancing. Thus, improved systems and methods for collecting, analyzing, and classifying image data and/or ligament laxity information are desired to help in planning surgeries, facilitate preoperative and intraoperative review of patient data, and improving patient outcomes.

### BRIEF SUMMARY OF THE DISCLOSURE

In some aspects, the techniques described herein relate to a computer-implemented method for generating and presenting a display of a visualization of joint parameters, the method including: receiving, by one or more processors, imaging data of a joint; extracting, by the one or more processors, gap parameter information from the imaging data, calculating, based on the gap parameter information, delta parameter information; generating, by the one or more processors, a graphical user interface (GUI) based on the delta parameter information, wherein the GUI includes a three-dimensional element configured to represent the delta parameter information; displaying, by the one or more processors, the generated GUI on an electronic display.

In some aspects, the techniques described herein relate to a system for generating and presenting a display of a visualization of joint parameters, including: a computer-readable storage medium storing instructions for generating and presenting a display of guidance for performing the robotic medical procedure; and one or more processors configured to execute the instructions to perform a method including: receiving imaging data of a joint; determining gap parameter information based on the imaging data, generating a graphical user interface (GUI) configured to display a three-dimensional element including a plurality of sub-elements; determining which of the plurality of sub-elements correspond to the determined gap parameter information; and generating a display of the GUI, wherein at least one sub-element is associated with the determined gap parameter information.

In some aspects, the techniques described herein relate to a computer-implemented method for generating a display for visualization of joint parameters, the method including: receiving, by one or more processors, imaging data of a joint; extracting, by the one or more processors, gap parameter information from the imaging data; calculating, by the one or more processors, based on the gap parameter information, delta parameter information; displaying, by the one or more processors, the delta parameter information on a graphical user interface (GUI).

### BRIEF DESCRIPTION OF THE DRAWINGS

A more complete appreciation of the subject matter of this disclosure and the various advantages thereof may be understood by reference to the following detailed description, in which reference is made to the following accompanying drawings:

FIG. 1 illustrates an exemplary knee joint in flexion and extension, according to aspects of this disclosure.

FIG. 2 illustrates a cross-sectional view of an exemplary knee joint including implant components, according to aspects of this disclosure.

FIG. 3 illustrates an exemplary electronic data processing system including a bone balancing system, according to an exemplary embodiment.

FIG. 4 illustrates exemplary process flow diagram including data, inputs, and outputs of the bone balancing system of FIG. 3, according to aspects of this disclosure.

FIG. 5 illustrates an exemplary 2-dimensional depiction of ligament laxity data, according to aspects of this disclosure.

FIG. 6 illustrates an exemplary 3-dimension depiction of ligament laxity data, according to aspects of this disclosure.

FIG. 7A is an exploded view of the 3-dimensional depiction of ligament laxity data along a first axis, according to aspects of this disclosure.

FIG. 7B is an exploded view of the 3-dimensional depiction of ligament laxity data along a second axis, according to aspects of this disclosure.

FIG. 7C is an exploded view of the 3-dimensional depiction of ligament laxity data along a third axis, according to aspects of this disclosure.

FIG. 8 illustrates an exemplary graphical user interface of the bone balancing system of FIG. 3, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc., or portion thereof. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means away from the center of the body, and the term “proximal” means close to the center of the body, for example the shoulder is a proximal part of the arm and the fingertips are a distal part of the arm. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Surgeries incorporating prosthetics and/or implants, such as joint replacement procedures, often require careful consideration of various factors. An important factor for implant planning in, for example, a knee replacement is soft tissue balance assessment. The standard gap quadrants used for soft tissue balance assessment in total knee replacements are made up of four sectors: medial extension, lateral extension, medial flexion, and lateral flexion. Analyzing and classifying image data and ligament laxity information for all four sectors in a 2-dimensional visualization provides a challenge. The present invention is directed to addressing this challenge by providing a 3-dimension visualization of the four sectors in a single image.

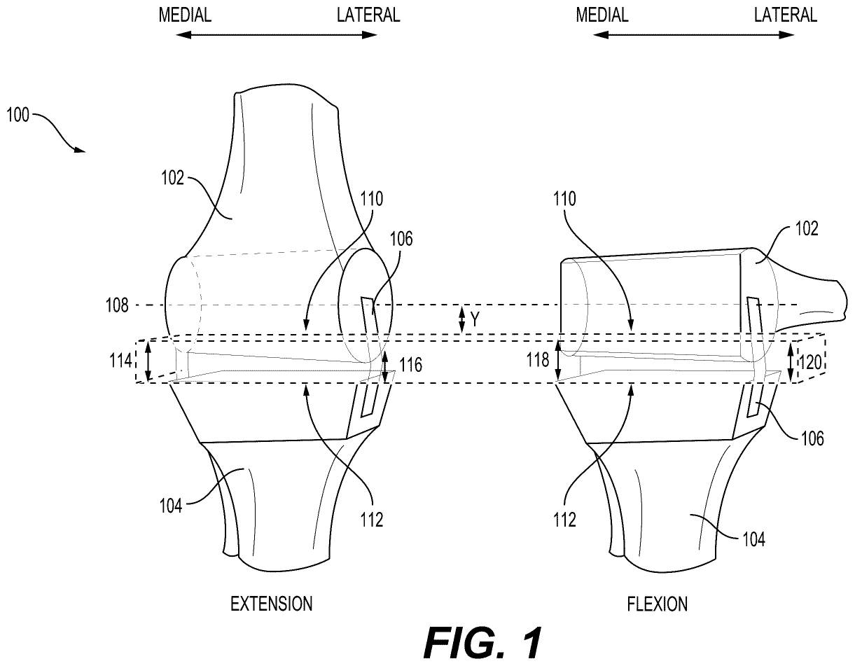

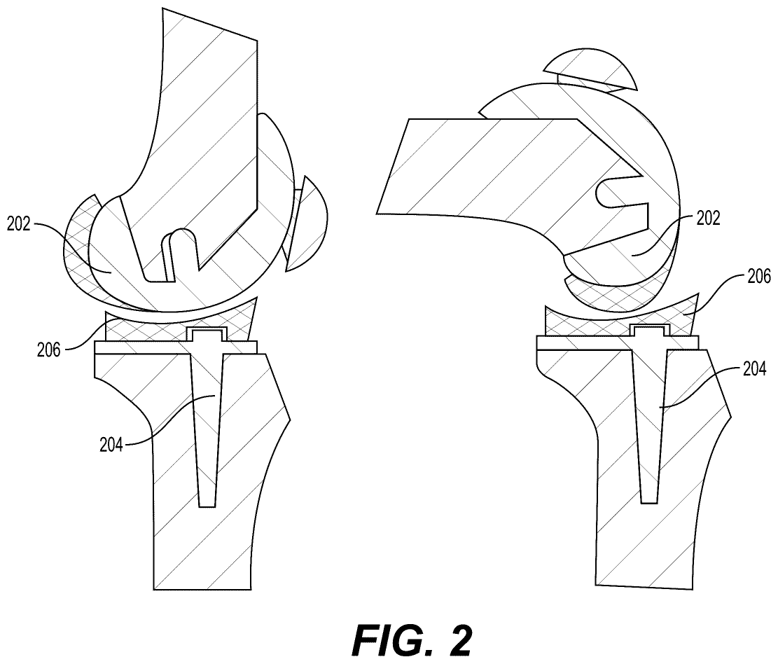

FIG. 1 illustrates an exemplary knee joint 100 in flexion and extension. Referring to FIG. 1, the knee joint 100 may include a femur 102, a tibia 104, and a ligament 106 (e.g. lateral collateral ligament) coupled to the femur 102 and the tibia 104. The femur 102 may rotate with respect to tibia 104 about a flexion-extension axis 108 extending through the femur 102. A procedure plan (e.g, surgery plan or medical operation plan) may include one or more bone cuts, such as a distal and posterior femoral resection 110 through the femur 102 and/or a proximal tibial resection 112 through the tibia 104. Note resection planes 110 and 112 are shown in dotted lines in FIG. 1. When the knee joint 100 is in extension, a medial extension gap 114 may extend between a medial end of the femoral resection 110 and the tibial resection 112, and a lateral extension gap 116 may extend between a lateral end of the femoral resection 110 and the tibial resection 112. When the knee joint 100 is in flexion, a medial flexion gap 118 may extend between a medial end of the femoral resection 110 and the tibial resection 112, and a lateral flexion gap 120 may extend between the lateral end of the femoral resection 110 and the tibial resection 112. The femoral resection 110 and the tibial resection 112 may be configured to provide a balanced knee joint 100, and in some examples, in conjunction with one or more prosthetic components, liners, or other implantable devices, such as the femoral prosthetic component 202, the tibial prosthetic component 204, and the liner 206 shown in FIG. 2. In some examples, the tibial prosthetic component 204 may include a tibial stem XXX (please add identifier in FIG. 2) configured to extend longitudinally through a tibia, for example through the medullary canal of a tibia.

When planning for a medical procedure at the knee joint 100, a practitioner (e.g., surgeon, doctor, healthcare planner, etc.) may desire to make the extension gap 114 equal to the flexion gap 116. In some examples, the practitioner may plan to create a rectangular flexion gap 116 by adjusting a femoral cut position so that the femoral cut position is parallel to a resected tibial surface at 90° with the ligaments (e.g., ligament 106) under tension.

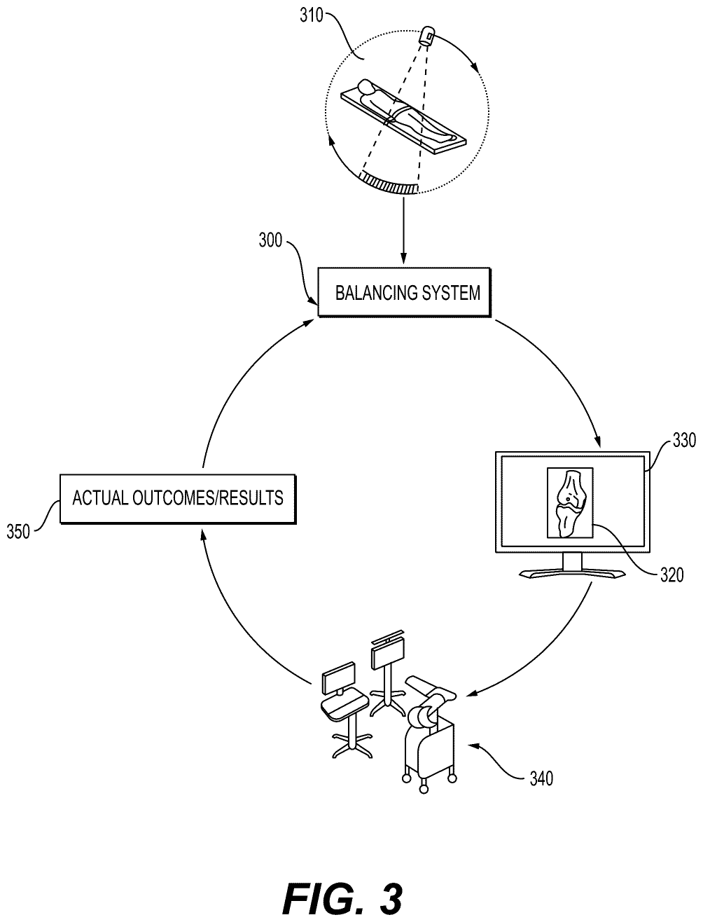

Referring to FIG. 3, an electronic data processing system 6 may include a bone balancing system 300 including one or more algorithms. The bone balancing system 300 may receive one or more medical images of patient's anatomy acquired using one or more image acquisition devices 310, analyze the received one or more medical images to determine and/or adjust a procedure plan 320, which may be output into a display 330 and/or to a robotic and/or automated data system or platform 310 (e.g., a robotic system such as a surgical robot and/or a robotic tool). Results and/or outcome data 350 from the procedure may be fed to the bone balancing system 300 for further refinement of the one or more algorithms.

The bone balancing system 300 may be implemented as one or more computer systems or cloud-based electronic processing systems, which may be incorporated into a surgical robot or robotic tool, or separate from a surgical robot or robotic tool. The image acquisition device 310 may include a computed tomography (CT) scanner, a magnetic resonance imaging (MRI) machine, an x-ray machine, a radiography system, an ultrasound system, a thermography system, a tactile imaging system, an electrography, nuclear medicine functional imaging system, a positron emission tomography (PET) system, a single-photon emission computer tomography (SPECT) system, a camera, etc. An instant patient who is planning to undergo a procedure (e.g., surgery) may first undergo imaging using the image acquisition device 310 (e.g., a CT scanner). Images and/or information collected during imaging (e.g., CT or CAT scans) may be transmitted from or stored in the image acquisition device 310.

During imaging using the image acquisition device 310, the patient may undergo one or more “poses” or positions where certain soft tissues of a joint (e.g., medial and/or lateral soft tissues) are stressed in certain positions of the joint (e.g., flexion and extension) by applying one or more forces (e.g., varus and valgus forces). The bone balancing system 300 may determine and/or calculate a size and/or shape of one or more gaps of the joint (e.g., flexion and extension gaps) to assess the soft tissue envelope of the joint, for example using images obtained by the image acquisition device 310 during the one or more poses. Alternatively or in addition thereto, the practitioner may determine a size and/or shape of the one or more gaps, and input the determined size and/or shape into the bone balancing system 300 (e.g., via a user interface such as display 330). The bone balancing system 300 may identify bone landmarks from the images and/or data (e.g., osteophytes and their dimensions and/or positions, contact point locations between two bones, etc.) collected at the poses. Alternatively, a practitioner may analyze the images and input, into the bone balancing system 300, imaging data, such as positions and dimensions of bone landmarks (e.g., osteophytes or other parameters).

The bone balancing system 300 may execute the one or more algorithms, using the imaging data including the determined gap (e.g., flexion and extension gap) to determine the procedure plan 320. The procedure plan 320 may include a series of steps to perform in the procedure, such as one or more bone resection parameters (e.g., resections or cuts to make in a bone and/or one or more osteophytes to remove), one or more bone alignment parameters (e.g. alignment of bone landmarks on different bones, etc.) and/or a design of an implant (e.g., prosthetic liner). The procedure plan 320 may also include predicted outcomes (e.g., a risk of complication during the procedure or a risk of infection post-procedure). As an example, the procedure plan 320 may first be determined based on the size and/or the shape of the one or more gaps of the joint, and then may be revised based on identified osteophytes that would be planned to be removed during the procedure.

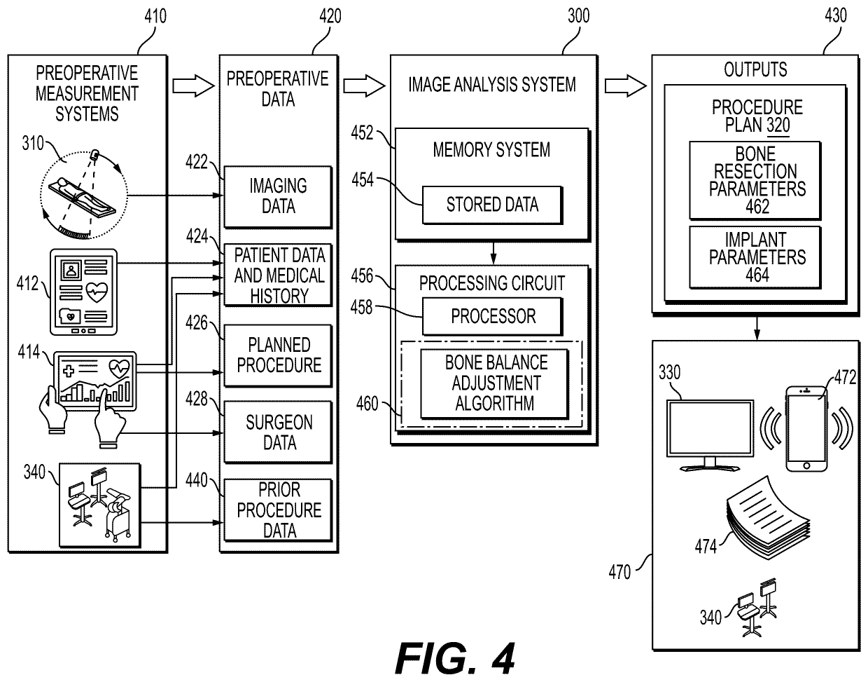

In some examples, the procedure plan 320, including the one or more bone resection parameters 462 and/or implant parameters 464 (FIG. 4), may be transmitted to a display 330. The one or more bone parameters 462 and the one or more implant parameters 464 of the procedure plan 320 may collectively be referred to as one or more adjustment parameters. In some examples, the procedure plan 320, including the one or more bone resection parameters 462 and/or implant parameters 464, may be transmitted to a surgical robot or robotic tool (e.g., an automated cutting burr, a Mako SmartRobotics™ surgical robot, etc.) of the robotic and/or automated data system 310 to execute the determined bone resection parameters 462 by, for example, automatically cutting, via a surgical robot holding a tool or a surgeon holding a robotic tool, etc., a bone according to the bone resection parameters 462 of the procedure plan 320. In some examples, the procedure plan 320 may include executing the determined bone resection parameters 462 via a practitioner operating a surgical robot, such as guiding a robotic arm with the assistance of the surgical robot (haptically, movement limiting, or otherwise assisting the practitioner). As the course of treatment is continued, the actual or observed outcomes and/or results 350 may also be used by the bone balancing system 300 to either update its predictions (e.g., intraoperatively based on intraoperatively collected data or outcomes) and/or to make future predictions for future patients (e.g., based on a postoperative result or outcome). Intraoperative data for further refinements may be similar to the preoperative data 420. Details of the bone balancing system 300 and its determinations will be described in more detail with respect to FIG. 4.

FIG. 4 illustrates an exemplary process flow diagram with types of data, inputs, and outputs of the bone balancing system 300. Referring to FIG. 4, the bone balancing system 300 may receive preoperative data 420 from one or more preoperative measurement systems 410 and analyze the preoperative data 420 to produce one or more outputs 430, such as the procedure plan 320, to one or more output systems 470, such as the display 330. The preoperative measurement systems 410 may include the image acquisition device 310; electronic devices storing electronic medical records (EMR) 414; patient, practitioner, and/or user interfaces or applications 450 (such as on tablets, computers, or other mobile devices); and the robotic and/or automated data system or platform 310 (e.g., Mako SmartRobotics™ surgical robot or platform, MakoSuite, etc.), which may include a robotic device, as previously mentioned.

The bone balancing system 300 may receive imaging data 422 via the image acquisition device 310 and supplemental or additional information (e.g., patient data and medical history 424, planned procedure data 426, surgeon and/or staff data 428, and/or prior procedure data 440) via EMR 412, interfaces 414, sensors and/or electronic medical devices, and/or robotic platform 310. Each of the devices in the preoperative measurement systems 410 (the image acquisition device 310, EMR 412, user interfaces or applications 414, sensors and/or electronic medical devices, and robotic platform 310) may include one or more communication modules (e.g., WiFi modules, BlueTooth modules, etc.) configured to transmit preoperative data 420 to each other, to the bone balancing system 300, and/or to the one or more output systems 470.

The image acquisition device 310 may be configured to collect or acquire one or more images, videos, and/or scans of a patient's internal anatomy, such as bones, ligaments, soft tissues, brain tissue, etc. to provide imaging data 422 to bone balancing system 300, which will be described in more detail later. As previously described, the image acquisition device 310 may include a computed tomography (CT) scanner, a magnetic resonance imaging (MRI) machine, an x-ray machine, a radiography system, an ultrasound system, a thermography system, a tactile imaging system, an electrography, nuclear medicine functional imaging system, a positron emission tomography (PET) system, a single-photon emission computer tomography (SPECT) system, a camera, etc. The collected images, videos, and/or scans may be transmitted, automatically or manually, to the bone balancing system 300. In some examples, a user may select specific images from a plurality of images taken with the image acquisition device 310 to be transmitted to the bone balancing system 300. In some examples, a filtering algorithm may be utilized to process a plurality of images and only send a sub-set of the processed images to the bone balancing system 300.

The bone balancing system 300 may use previously collected data from EMR 412, which may include patient data and medical history 424 in the form of past practitioner assessments, medical records, past patient reported data, past imaging procedures, treatments, etc. For example, EMR 412 may contain data on demographics, medical history, biometrics, past procedures, general observations about the patient (e.g., mental health), lifestyle information, data from physical therapy, etc.

The bone balancing system 300 may also use current (e.g., in real time) patient data via patient, practitioner, and/or user interfaces or applications 414. These user interfaces 414 may be implemented on mobile applications and/or patient management websites or interfaces, such as OrthologIQ®. User interfaces 414 may present questionnaires, surveys, or other prompts for practitioners or patients to enter assessments (e.g., throughout a prehabilitation program prior to a procedure), observed data and/or reactions (e.g., in response to various poses), psychosocial information and/or readiness for surgery, comments, etc. for additional patient data 424. Patients may also enter psychosocial information such as perceived or evaluated pain, stress level, anxiety level, feelings, and other patient reported outcome measures (PROMS) into these user interfaces 414. Patients and/or practitioners may report lifestyle information via user interfaces 414. User interfaces 414 may also collect clinical data such as planned procedure 426 data and planned surgeon and/or staff data 428 described in more detail later. These user interfaces 414 may be executed on and/or combined with other devices disclosed herein (e.g., with robotic platform 310).

The bone balancing system 300 may receive prior procedure data 440 from prior patients and/or other real-time data or observations (e.g., observed patient data 424) via robotic platform 310. The robotic platform 310 may include one or more robotic devices (e.g., surgical robot), computers, databases, etc. used in prior procedures with different patients. The robotic platform 310 may have assisted with, via automated movement, surgeon assisted movement, and/or sensing, a prior procedure and may be implemented as or include one or more automated or robotic surgical tools, robotic surgical or Computerized Numerical Control (CNC) robots, surgical haptic robots, surgical tele-operative robots, surgical hand-held robots, or any other surgical robot.

Although the preoperative measurement system(s) 410 is described in connection with image acquisition device 310, EMR 412, user interfaces 414, and robotic platform 310, other devices may be used preoperatively to collect preoperative data 420, for example data relating to joint alignment and/or identified bone landmarks and/or osteophytes and/or other data may be used to create procedure plan 320. For example, mobile devices such as cell phones and/or smart watches may include various sensors (e.g., gyroscopes, accelerometers, temperature sensors, optical or light sensors, magnetometer, compass, global positioning systems (GPS) etc.) to collect patient data 424 such as location data, sleep patterns, movement data, heart rate data, lifestyle data, activity data, etc. As another example, wearable sensors, heart rate monitors, motion sensors, external cameras, etc. having various sensors (e.g., cameras, optical light sensors, barometers, GPS, accelerometers, temperature sensors, pressure sensors, magnetometer or compass, MEMs devices, inclinometers, acoustical ranging, etc.) may be used during physical therapy or a prehabilitation program to collect information on patient kinematics, alignment, movement, fitness, heart rate, electrocardiogram data, breathing rate, temperature, oxygenation, sleep patterns, activity frequency and intensity, sweat, perspiration, air circulation, stress, step pressure or push-off power, balance, heel strike, gait, fall risk, frailty, overall function, etc. Other types of systems or devices may include electromyography or EMG systems or devices, motion capture (mocap) systems, sensors using machine vision (MV) technology, virtual reality (VR) or augmented reality (AR) systems, etc.

The preoperative data 420 may be data collected, received, and/or stored prior to an initiation of a medical treatment plan or medical procedure. As shown by the arrows in FIG. 4, the preoperative data 420 may be collected using the preoperative measurement systems 410, from a memory system 452 (e.g., cloud storage system) of the bone balancing system 300, and from output systems 470 (e.g., from a prior procedure) for one or more continuous feedback loops. Some of the preoperative data 424 may be directly sensed via one or more devices (e.g., image acquisition device 310 and/or wearable motion sensors or mobile devices) or may be manually entered by a medical professional, patient, or other party. Other preoperative data 424 may be determined (e.g., by bone balancing system 300) based on directly sensed information, input information, and/or stored information from prior medical procedures.

As previously described, the preoperative data 424 may include imaging data 422, patient data and/or medical history 424, information on a planned procedure 426, surgeon data 428, and prior procedure data 440.

The imaging data 422 may include one or more images (e.g., raw images), videos, or scans of a patient's anatomy collected and/or acquired by the image acquisition device 310. The bone balancing system 300 may receive and analyze one or more of these images to determine further imaging data 422, which may be used as further input preoperative data 424. In some example, image acquisition device 310 may analyze and/or process the one or more images, and send any analyzed and/or processed imaging data to the bone balancing system 300 for further analysis.

The one or more images of the imaging data 422 may illustrate or otherwise indicate, and the bone balancing system 300 may be configured to identify and/or recognize in the images: bone, osteophyte, soft tissue, or cartilage positions and/or alignment, composition and/or density, fractures and/or tears, bone landmarks (e.g., condyle surface, head or epiphysis, neck or metaphysis, body or diaphysis, articular surface, epicondyle, lateral epicondyle, medial epicondyle, process, protuberance, tubercle vs tuberosity, tibial tubercle, trochanter, spine, linea or line, facet, crests and ridges, foramen and fissure, meatus, fossa and fovea, incisure and sulcus, and sinus), geometry (e.g., diameters, slopes, angles) and/or other anatomical geometry data such as deformities or flare (e.g., coronal plane deformity, sagittal plane deformity, lateral femoral metaphyseal flare, or medial femoral metaphyseal flare). Such geometry is not limited to overall geometry and may include relative dimensions (e.g., lengths or thicknesses of a tibia or femur).

The imaging data 422 may indicate and/or be used to determine osteophyte size, volume, and/or positions; bone loss; joint space; B-score; bone quality/density; skin-to-bone ratio; bone loss; hardware detection; anterior-posterior (AP) and medial-lateral (ML) distal femur size, and/or joint angles. Analysis and/or calculations that may be derived from the images will be described in more detail below when describing the bone balancing system 300.

In addition, the imaging data 422 may include morphology and/or anthropometrics (e.g., physical dimensions of internal organs, bones, etc.), fractures, slope or angular data, tibial slope, posterior tibial slope or PTS, bone density, (e.g., bone mineral or bone marrow density, bone softness or hardness, or bone impact), etc. Imaging data 422 may not be limited to bone data and may be inclusive of other internal imaging data, such as of cartilage, soft tissue, blood flow, or ligaments.

In addition to raw images, imaging data 422 may include intermediate and/or related imaging data 422 to be used by the bone balancing system 300 to calculate outputs 430. Such intermediate imaging data 422 may include density or composition charts or graphs; quantified data indicating relative positions, dimensions, etc.; and/or processed image data indicating specifically detected attributes, such as a probability of a certain patient condition. One or more algorithms 460 of the bone balancing system 300 may determine or calculate this intermediate imaging data 422 in determining outputs 430, or alternatively or additionally thereto, the image acquisition device 310 may include one or more processors configured to calculate or quantify, based on the raw images, videos, or scans, at least some of the intermediate imaging data 422. Intermediate imaging data 422 may include information relating to, indicating, and/or quantifying aspects of the raw images, charts, etc.

Patient data and medical history 424 may include information about the instant patient on identity (e.g., name or birthdate), demographics (e.g., patient age, gender, height, weight, nationality, body mass index (BMI), etc.), lifestyle (e.g., smoking habits, exercise habits, drinking habits, eating habits, fitness, activity level, frequency of climbing activities such as up and down stairs, frequency of sit-to-stand movements or bending movements such as when entering and exiting a vehicle, steps per day, activities of daily living or ADLs performed, etc.), medical history (e.g., allergies, disease progressions, addictions, prior medication use, prior drug use, prior infections, frailties, comorbidities, prior surgeries or treatment, prior injuries, prior pregnancies, utilization of orthotics, braces, prosthetics, or other medical devices, etc.), assessments and/or evaluations (e.g., laboratory tests and/or bloodwork, American Society of Anesthesiology or ASA score and/or fitness for surgery or aesthesia) electromyography data (muscle response or electrical activity in response to a nerve's stimulation), psychosocial information (e.g., perceived pain, stress level, anxiety level, mental health status, PROMS (e.g., knee injury and osteoarthritis outcome score or KOOS, hip disability and osteoarthritis outcome score or HOOS, pain virtual analog scale or VAS, PROMIS Global 10 or PROMIS-10, EQ-5D, a mental component summary, satisfaction or expectation information, etc.), past biometrics (e.g., heart rate or heat rate variability, electrocardiogram data, breathing rate, temperature (e.g., internal or skin temperature), fingerprints, DNA, etc.), past kinematics or alignment data, past imaging data, data from prehabilitation programs or physical therapy (e.g., average load bearing time) etc. Medical history 424 may include prior clinical or hospital visit information, including encounter types, dates of admission, hospital-reported comorbidity data such as Elixhauser and/or Charlson scores or selected comorbidities (e.g., ICD-10 POA), prior anesthesia taken and/or reactions, etc. This list, however, is not exhaustive and preoperative data 420 may include other patient specific information, clinical information, and/or surgeon or practitioner specific information (e.g., experience level).

Patient data 424 may come from EMR 412, user interfaces 414, from memory system 452, and/or from robotic platform 310, but aspects disclosed herein are not limited to a collection of the patient data 424. For example, other types of patient data 424 or additional data may include data on activity level; kinematics; muscle function or capability; range of motion data; strength measurements and/or force measurements push-off power, force, or acceleration; a power, force, or acceleration at a toe during walking; angular range or axes of joint motion or joint range of motion; flexion or extension data, including step data (e.g., measured by a pedometer), gait data or assessments; fall risk data; balancing data; joint stiffness or laxity data; postural sway data; data from tests conducted in a clinic or remotely; etc.

Information on a planned procedure 426 may include an initial procedure plan 320 and/or logistical information about the procedure and substantive information about the procedure. Substantive planned procedure 426 information may include a surgeon's surgical or other procedure or treatment plan, including planned steps or instructions on incisions, a side of the patient's body to operate on (e.g., left or right) and/or laterality information, bone cuts or resection depths, implant design, type, and/or size, implant alignment, fixation or tool information (e.g., implants, rods, plates, screws, wires, nails, bearings used), cementing versus cementless techniques or implants, final or desired alignment, pose or orientation information (e.g., capture gap values for flexion or extension, gap space or width between two or more bones, joint alignment), planning time, gap balancing time, extended haptic boundary usage, etc. Logistical planned procedure 426 information may include information about a planned site of the procedure such as a hospital, a type of procedure or surgery to be performed (e.g., total or partial knee arthroplasty or replacement, total or partial hip arthroplasty or replacement, spine surgery, patella resurfacing), scheduling or booking information, equipment or tools required, etc. This initial planned procedure 426 information may be manually prepared or input by a surgeon and/or previously prepared or determined using one or more algorithms. Surgeon data 428 may include information about a surgeon or other staff planned to perform the procedure plan 320 such as identity (e.g., name), experience level, fitness level, height and/or weight, etc.

Prior procedure data 440 may include information about prior procedures performed on a same or prior patient. Such information may include the same type of information as in planned procedure data 428 (e.g., instructions or steps of a procedure, bone cuts, implant design, implant alignment, etc.) along with outcome and/or result information (e.g., outcomes 350 described with reference to FIG. 3), which may include both immediate results and long-term results, complications after surgery, length of stay in a hospital, revision surgery data, rehabilitation data, patient motion and/or movement data, etc. Prior procedure data 440 may include information about prior procedures of prior patients sharing at least one same or similar characteristic (e.g., demographically, biometrically, disease state, etc.) as the instant patient.

Preoperative data 420 may include any other additional or supplemental information stored in memory system 452, which may also include known data and/or data from third parties, such as data from the Knee Society Clinical Rating System (KSS) or data from the Western Ontario and McMaster Universities Osteoarthritis Index (WOMAC). In addition, in some examples, intraoperative may be collected and/or received by the bone balancing system 300 to further generate and/or refine the outputs 430 (e.g., procedure plan 320).

### The Bone Balancing System 300

The bone balancing system 300 may include a memory system 452 including stored data 454 and a processing circuit 456 including a processor 458 and one or more algorithms. The one or more algorithms may include a bone balance adjustment algorithm 460. The bone balance adjustment algorithm 460 may use one or more linear relationships to analyze the imaging data 422 to generate and/or modify the procedure plan 320. In some examples, the bone balancing system 300 may include an artificial intelligence (Al) and/or machine learning system that is “trained” or that may learn and refine the relationships, patterns, etc. between preoperative data 420, outputs 430, and actual results 350 (FIG. 3) to make determinations and/or, in some examples, to refine the bone balance adjustment algorithm 460. The bone balancing system 300 and the bone balance adjustment algorithm 460 may alternatively be referred to as a bony balancing system 300 and a bony balance adjustment algorithm 460 and/or a gap or soft tissue balancing system 300 or a gap or soft tissue balance adjustment algorithm 460.

The bone balancing system 300 may be implemented using one or more computing platforms, such as platforms including one or more computer systems and/or electronic cloud processing systems. Examples of one or more computing platforms may include, but are not limited to, smartphones, wearable devices, tablets, laptop computers, desktop computers, Internet of Things (IoT) device, remote server/cloud based computing devices, or other mobile or stationary devices. The bone balancing system 300 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The bone balancing system 300 may include one or more communication modules (e.g.., WiFi or Bluetooth modules) configured to communicate with preoperative measurement systems 422, output system 470, and/or other third-party devices, etc. For example, such communication modules may include an Ethernet card and/or port for sending and receiving data via an Ethernet-based communications link or network, or a Wi-Fi transceiver for communication via a wireless communications network. Such communication modules may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). Such communication modules may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink).

The memory system 452 may have one or more memories or storages configured to store or maintain the preoperative data 420, outputs 430, and stored data 454 from prior patients and/or prior procedures. The preoperative data 420 and outputs 430 of an instant procedure may also become stored data 454, for example for use in developing procedure plans for a future procedure. Although the memory system 452 is illustrated close to processing circuit 456, memory system 452 may include memories or storages implemented on separate circuits, housings, devices, and/or computing platforms and in communication with bone balancing system 300, such as cloud storage systems and other remote electronic storage systems.

The memory system 452 may include one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein.

The memory system 20 may include volatile memory or non-volatile memory (e.g., semiconductor-based memory device, a magnetic memory device and system, an optical memory device and system, fixed memory, or removable memory). The memory system 452 may include database components, object code components, script components, or any other type of information structure to support the various activities described herein. In some aspects, the memory system 452 may be communicably connected to the processing circuit 456 and may include computer code to execute one or more processes described herein. The memory system 452 may contain a variety of modules, each capable of storing data and/or computer code related to specific types of functions.

The processing circuit 456 may include a processor 458 configured to execute or perform the one or more algorithms, including the bone balance adjustment algorithm 460, based on received data, which may include the preoperative data 420 and/or any data in the memory system 452 to determine the outputs 430. The preoperative data 420 may be received via manual input, retrieved from the memory system 452, and/or received direction from the preoperative measurement systems 422. The processor 458 may be configured to determine patterns based on the received data.

The processor 458 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 458 may be configured to perform machine readable instructions, which may include one or more modules implemented as one or more functional logic, hardware logic, electronic circuitry, software modules, etc. In some cases, the processor 458 may be remote from one or more of the computing platforms comprising the bone balancing system 300. The processor 458 may be configured to perform one or more functions associated with the bone balancing system 300, such as precoding of antenna gain/phase parameters, encoding and decoding of individual bits forming a communication message, formatting of information, and overall control of one or more computing platforms comprising the bone balancing system 300, including processes related to management of communication resources and/or communication modules.

In some aspects, the processing circuit 456 and/or memory system 452 may contain several modules related to medical procedures, such as an input module, an analysis module, and an output module. The bone balancing system 300 need not be contained in a single housing. Rather, components of the bone balancing system 300 may be located in various different locations or even in a remote location. Components of the bone balancing system 300, including components of the processing circuit 456 and the memory system 452, may be located, for example, in components of different computers, robotic systems, devices, etc. used in surgical procedures.

The bone balancing system 300 may use the one or more algorithms, including the bone balance adjustment algorithm 460, to make intermediate determinations and to determine the one or more outputs 430. The one or more algorithms may be configured to determine or glean data from the preoperative data 420, including the imaging data 422. For example, the one or more algorithms may be configured for bone recognition, soft tissue recognition, and/or to make determinations related to the intermediate imaging data 422 previously described. The one or more algorithms may operate simultaneously and/or separately to determine the one or more outputs 430 and/or display the one or more outputs 430.

The one or more algorithms include the bone balance adjustment algorithm 460. In addition, the one or more algorithms may include one or more machine learning algorithms that are trained using, for example, linear regression, random forest regression, CatBoost regression, statistical shape modelling or SSM, etc. The one or more algorithms may be continuously modified and/or refined based on actual outcomes and/or results 350 (FIG. 3). The one or more algorithms may be configured to use segmentation techniques and/or thresholding techniques on received images, videos, and/or scans of the imaging data 422 to determine the one or more outputs 422. For example, the one or more algorithms may be configured to segment an image (e.g., a CT scan) and/or threshold soft tissue or identified bone, bone landmarks, osteophytes, etc. The one or more algorithms may be configured to automate data extraction and/or collection upon receiving an image from the image acquisition device 310. The bone balance adjustment algorithm 460 will be described in more detail hereinafter.

The one or more outputs 430 may include the procedure plan 320, but are not limited thereto. For example, the one or more outputs 430 may include related graphics, texts, or graphical user interfaces (GUIs) to display the procedure plan 320, patient anatomy representations, determined and/or enhanced images, etc. configured to be displayed on the display 330 or other output systems 470. Each of these outputs 430 may be used as input preoperative data 422 to determine other outputs 430.

The outputs 430 may be output to any one or more of the output systems 470 via a wireless, electronic, and/or wired connection. The output systems 470 may include the display 330, a mobile device 442, or physical paper, canvas, film or other physical medium 444.

### The Bone Balance Adjustment Algorithm 460

To determine the one or more bone resection parameters 462 and/or implant parameters 464, the bone balancing system 300 may execute the bone balance adjustment algorithm 460. The bone balance adjustment algorithm 460 may be one algorithm and/or may include multiple algorithms that perform different calculations (e.g., one algorithm for each type of bone cut, soft tissue envelope, etc.) and/or that perform related functions (e.g., bone recognition, surface area calculation, and bone parameter determination). For convenience of description, the bone balance adjustment algorithm 460 will be described as one algorithm. Although the bone balancing system 300 is described as executing the bone balance adjustment algorithm 460, aspects disclosed herein are not limited to a device or system that executes the bone balance adjustment algorithm 460.

As previously described, the bone balance adjustment algorithm 460 may be configured to determine the one or more bone resection parameters 462 and/or one or more implant parameters 464 for a joint based on identified osteophytes, identified location and/or dimensions of the osteophytes, and their planned removal. The bone resection parameters 462 may include a laxity parameter, a bone cut adjustment, a bone cut depth, a bone cut angle or slope, or other adjustments a surgeon may make during a procedure where bone resections are made. The implant parameters 464 may include one or more dimensions of an implant configured to balance the joint and/or a gap of the joint, such as an implant thickness.

For example, the bone balance adjustment algorithm 460 may be configured to predict an amount of soft tissue laxity that will be created by removing identified osteophytes based on a size and/or location of the osteophyte before they are removed. For example, when posterior osteophytes are present on a femur or a tibia, a practitioner may plan to remove them. Due to their posterior location, access to posterior osteophytes may only be possible after tibial and femoral bone cuts are made. Once the osteophytes are removed, the soft tissue envelope may change, effectively changing both the size and shape of flexion and extension gaps, rendering the pre-resection soft tissue “poses” inaccurate. Thus, the bone balance adjustment algorithm 460 may predict a change in laxity of the soft tissue envelope, and adjust (e.g., reduce) the bone resection parameters 462, implant parameters 464, and/or other parameters of the procedure plan 320.

The bone balance adjustment algorithm 460 may be configured to anticipate the laxity created by osteophyte removal, and may determine an alteration of bone resection parameters 462 and/or implant parameters 464 (e.g., thickness of a tibial liner, recommendation for an implant, etc.) to accommodate the increased laxity. The bone balance adjustment algorithm 460 may analyze a predetermined (e.g., sagittal) view of a joint (e.g., knee joint), identify, based on imaging data and/or acquired images of a sagittal view (or other view) using the image acquisition device 310, one or more osteophytes that may not be readily removed before making bone cuts (e.g., posterior osteophyte), determine a surface area of each identified osteophyte, and determine an amount of laxity based on the determined surface area.

The determination may be based on a learned relationship and/or a linear equation. As an example, the bone balance adjustment algorithm 460 may execute a simple y=mx+b formatted equation, where “x” is the surface area in mm²; “y” is the change in a bone resection depth in millimeters or mm, a change in bone resection angle or slope in degrees, or is the change in an implant thickness in mm; m is a multiplier (e.g., input by the practitioner, learned, and/or refined based on results); and b is a constant. For example, in the context of a knee joint, “b” may be 0 and/or adjusted based on preoperative data 420 (e.g., patient data 424, procedure plan 320), and “m” may be in a range of 0.004-0.006 (e.g., 0.005, 0.0055, or 0.0056), but aspects disclosed herein are not limited. For example, “m” and “b” may be different values for different joints and/or adjusted based on patient data. “M” and “b” may also be further adjusted based on learned relationships (e.g., gender) or other preoperative data 420 (e.g., cartilage condition or disease progression), predictions (e.g., cartilage loss or predicted disease progression), intraoperative data, etc. A sign of “m” may depend on an osteophyte symmetry and/or a type of parameter being adjusted. For example, where “y” indicates a bone resection depth, “m” may be negative to indicate a decrease in bone resection with respect to that of the initial procedure plan 320. Where “y” indicates an implant thickness, “m” may be positive to indicate an increase in implant thickness with respect to that of the initial procedure plan 320. Where “y” indicates a bone resection angle or slope, the sign of “m” may depend on whether an osteophyte at one side of the joint is greater than or less than an osteophyte at an opposite of the joint, as will be described in more detail hereinafter.

For example, the bone balance adjustment algorithm 460 may determine that the removal of an osteophyte having a relevant surface area of 90 mm² will result in 0.5 mm of soft tissue laxity based on a linear relationship of 0.5 mm per every 90 mm² and/or 0.1 mm per every 18 mm² (e.g., 180 mm² will result in 1.0 mm of soft tissue laxity, 240 mm² will result in 1.5 mm of soft tissue laxity, 18 mm² will result in 0.1 mm of soft tissue laxity, etc.). In some examples, the linear relationship may be simplified and/or rounded up to 0.5 mm per every 100 mm² and/or 0.1 mm per every 20 mm².

The bone balance adjustment algorithm 460 may be configured to determine a linear equation and/or refine the linear equation based on outcomes 350. For example, the bone balance adjustment algorithm 460 may multiply 0.0055 or 0.0056 by the surface area to determine a predicted laxity and/or change in bone resection parameter 462 and/or change in implant parameter 464, but aspects disclosed herein are not limited to this multiplier, which may be adjusted and refined. The bone balance adjustment algorithm 460 may also store an index of surface areas and adjustments (e.g., in a table or database), which may be updated and/or refined based on the outcomes 350.

The bone balance adjustment algorithm 460 may also consider a position of the osteophyte planned to be removed to predict the resulting laxity and/or direction of laxity in the soft tissue and/or other bone resection parameters 462. For example, if an osteophyte is removed from a posterolateral femur or a posterolateral tibia, then a soft tissue laxity may be lateral. If the osteophyte is removed from a posteromedial femur or a posteromedial tibia, then the soft tissue laxity may be medial. If there are symmetric posterolateral and posteromedial femoral or tibial osteophytes, then the soft tissue laxity may be global. If there are asymmetric posterolateral and posteromedial femoral or tibial osteophytes, then the soft tissue laxity may be asymmetric.

The bone resection parameters 462 may include angular adjustments and/or slopes determined based on the determined surface area of the osteophyte and/or the determined laxity. For example, if the bone balance adjustment algorithm 460 determines that an osteophyte of a joint is medial, the bone balance adjustment algorithm 460 may determine that removal of the medial osteophyte will create laxity of a medial gap of the joint in flexion and extension. Because some may disagree with the extent of laxity created in flexion in certain joints determined by the bone balance adjustment algorithm, in some examples, the bone balance adjustment algorithm 460 may be adjusted and/or consider a practitioner's preference and may determine that removal of the medial osteophyte will create laxity of the medial gap of the joint primarily in extension, but create less laxity (or none at all) in flexion, among other examples. For example, the bone balance adjustment algorithm 460 may change a type of bone to which less resection is recommended (e.g., tibia to femur).

In the context of a knee joint, the bone balance adjustment algorithm 460 may determine an alteration of a tibial bone cut (and/or a femoral bone cut, depending on a practitioner's preference) in the procedure plan 320 in anticipation of the determined laxity of the medial gap based on the determined surface area of the medial osteophyte. An angular adjustment may correspond to the predicted laxity. For example, if the bone balance adjustment algorithm 460 determines that the surface area of the medial osteophyte is 90 mm², the bone balancing system may determine a bone resection parameter 462 of 0.5° of tibial valgus (or 0.5° of tibial valgus per 0.5 mm of predicted laxity, or 0.1° of tibial valgus per 0.1 mm of predicted laxity) based on a linear relationship of 0.5° per every 90 mm² and/or 0.1° per every 20 mm² (e.g., 180 mm² will result in 1.0° of tibial valgus, 240 mm² will result in 1.5° of tibial valgus, 20 mm² will result in 0.1° of tibial valgus, etc.). For example, the bone balancing system 300 may multiply 0.0055 or 0.0056 by the surface area to determine a predicted angle or slope (e.g., tibial valgus), but aspects disclosed herein are not limited to this multiplier, which may be adjusted and refined. The bone balance adjustment algorithm 460 may also store an index of surface areas and adjustments, which may be updated and/or refined based on the outcomes 350.

Similarly, if the bone balance adjustment algorithm 460 determines that an osteophyte of a joint is lateral, the bone balance adjustment algorithm 460 may determine that removal of the lateral osteophyte will create laxity of a lateral gap of the joint in flexion and extension. Because some may disagree with the extent of laxity created in flexion, in some examples, the bone balance adjustment algorithm 460 may be adjusted and/or consider a practitioner's preference and may determine that removal of the medial osteophyte will create laxity of the medial gap of the joint primarily in extension, but create less laxity (or none at all) in flexion.

In the context of a knee joint, the bone balance adjustment algorithm 460 may determine an alteration of a tibial bone cut in the procedure plan 320 in anticipation of the determined laxity of the lateral gap based on the determined surface area of the lateral osteophyte. An angular adjustment may correspond to the predicted laxity. For example, if the bone balance adjustment algorithm 460 determines that the surface area of the lateral osteophyte is 90 mm², the bone balancing system may determine a bone resection parameter 462 of 0.5° of tibial varus (or 0.5° of tibial varus per 0.5 mm of predicted laxity) based on a linear relationship of 0.5° per every 90 mm² and/or 0.1° per every 20 mm² (e.g., 180 mm² will result in 1.0° of tibial varus, 240 mm² will result in 1.5° of tibial varus, 20 mm² will result in 0.1° of tibial varus). For example, the bone balance adjustment algorithm 460 may multiply 0.0055 or 0.0056 by the surface area to determine a predicted angle or slope (e.g., tibial varus), but aspects disclosed herein are not limited to this multiplier, which may be adjusted and refined. The bone balance adjustment algorithm 460 may also store an index of surface areas and adjustments, which may be updated and/or refined based on the outcomes 350.

If the bone balance adjustment algorithm 460 identifies symmetric osteophytes medially and laterally of a joint, then the bone balance adjustment algorithm 460 may determine that removal of these symmetric osteophytes will create global laxity in extension and/or flexion of the joint. In the context of a knee joint, the bone balance adjustment algorithm 460 may determine an alteration of a tibial bone in anticipation of the determined laxity based on the determined surface area of the medial osteophyte. For example, if the bone balance adjustment algorithm 460 determines that the surface area of each of the medial and lateral osteophytes are 90 mm², the bone balance adjustment algorithm 460 may determine a tibial resection of the procedure plan 320 to be 0.5 mm less tibial resection based on a linear relationship of 0.5 mm less tibial resection per every 90 mm² and/or 0.1 mm less tibial resection per every 20 mm²(e.g., 180 mm² will result in 1.0 mm less tibial resection, 240 mm² will result in 1.5 mm less of tibial resection, 20 mm² will result in 0.1 mm less of tibial resection, etc.). Alternatively, bone balance adjustment algorithm 460 may determine a liner used in the procedure plan 320 should have a thickness of 0.5 mm more and/or an increased thickness based on the linear relationship as an implant parameter 464.

If the bone balancing system 300 identifies medial and lateral osteophytes of different size, then the bone balance adjustment algorithm 460 may determine a greater laxity and/or adjustment to the procedure plan 320 (e.g., tibial bone cut) on a side having the larger osteophyte. For example, the bone balancing system 300 may determine a difference in a surface area of a medial osteophyte and a surface area of a lateral osteophyte. Where the medial osteophyte is larger than the lateral osteophyte and the difference in surface area is 90 mm², the bone balance adjustment algorithm 460 may determine that the procedure plan 320 should include a bone resection parameter 462 of 0.5° of tibial valgus based on a linear relationship of 0.5° per every 90 mm² and/or 0.1° per every 20 mm² (e.g., 180 mm² will result in 1.0° of tibial valgus, 240 mm² will result in 1.5° of tibial valgus, 20 mm² will result in 0.1° of tibial valgus, etc.). Similarly, where the lateral osteophyte is larger than the medial osteophyte and the difference in surface area is 90 mm², the bone balance adjustment algorithm 460 may determine that the procedure plan 320 should include a bone resection parameter 462 of 0.5° of tibial varus based on a linear relationship of 0.5° per every 90 mm² and/or 0.1° per every 20 mm² (e.g., 180 mm² will result in 1.0° of tibial varus, 240 mm² will result in 1.5° of tibial varus, 20 mm² will result in 0.1° of tibial valgus etc.). etc.

The bone balancing system 300 may be implemented as one or more computer systems or cloud-based electronic processing systems. The image acquisition device 310 may include a computed tomography (CT) scanner, a magnetic resonance imaging (MRI) machine, an x-ray machine, a radiography system, an ultrasound system, a thermography system, a tactile imaging system, an electrography, nuclear medicine functional imaging system, a positron emission tomography (PET) system, a single-photon emission computer tomography (SPECT) system, a camera, etc. An instant patient who is planning to undergo a procedure (e.g., surgery) may first undergo imaging using the image acquisition device 310 (e.g., a CT scanner). Images and/or information collected during imaging (e.g., CT or CAT scans) may be transmitted from or stored in the image acquisition device 310.

During imaging using the image acquisition device 310, the patient may undergo one or more “poses” or positions where certain soft tissues of a joint (e.g., medial and/or lateral soft tissues) are stressed in certain positions of the joint (e.g., flexion and extension) by applying one or more forces (e.g., varus and valgus forces). The bone balancing system 300 may determine and/or calculate a size and/or shape of one or more gaps of the joint (e.g., medial and lateral flexion and extension gaps) to assess the soft tissue envelope of the joint.

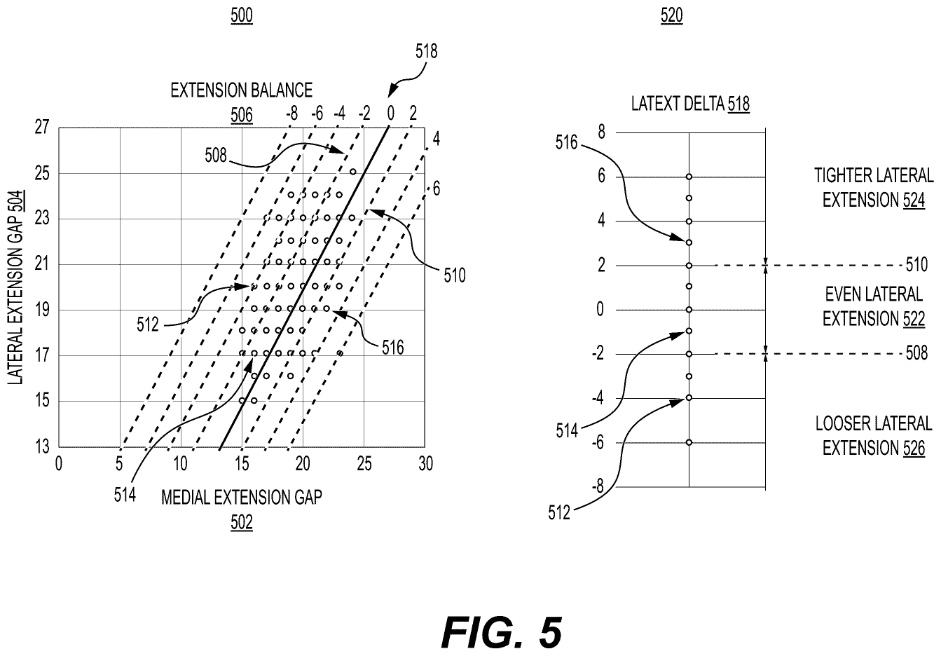

FIG. 5 illustrates an exemplary 2-dimensional depiction of ligament laxity data acquired and processed from the image acquisition device 310 for a number of patients. Chart 500 comprises a graph of the medial extension gap 114 for a number of patients along the x-axis 502 and the lateral extension gap 116 for the same set of patients along the y-axis 504, measured in millimeters. The diagonal lines in chart 500, such as line 508 and line 510, represent lines of equal difference, or delta, between the medial extension gap and the lateral extension gap. To illustrate, data point 512 represents a patient with a medial extension gap of 16 mm and a lateral extension gap of 20 mm. This data point falls along the diagonal line representing a delta of −4 mm, as the delta in this example is measured as delta=medial extension gap−lateral extension gap. Similarly, data point 514 has a medial extension gap of 16 mm and a lateral extension gap of 17 mm, for a lateral extension gap delta of −1 mm. As such, it may be noted that data point 514 represents a patient that has a nearly even delta between the lateral extension gap and the medial extension gap, while data point 512 represents a patient that has a looser lateral extension gap than medial extension gap. Data point 516 represents a patient with a lateral extension gap delta of +3 mm (delta=22 mm medial extension gap−19 mm lateral extension gap). This patient has a relatively tighter lateral extension delta.

Chart 520 compresses the 2-dimensional data of chart 500 into a single delta dimension, with a subset 522 of patients being determined to have substantially even lateral extension (i.e., a lateral extension delta between −2 and +2), a subset 524 of patients determined to have a tighter lateral extension delta (delta greater than 2), and a subset 526 of patients determined to have a looser lateral extension delta (delta less than −2). By compiling empirical data from a sample set of patients, a determination may be made as to where to draw the lines 508 and 510 between the subsets. This determination is based on empirical data that is useful to practitioners and may be updated based on changing data or feedback from practitioners.

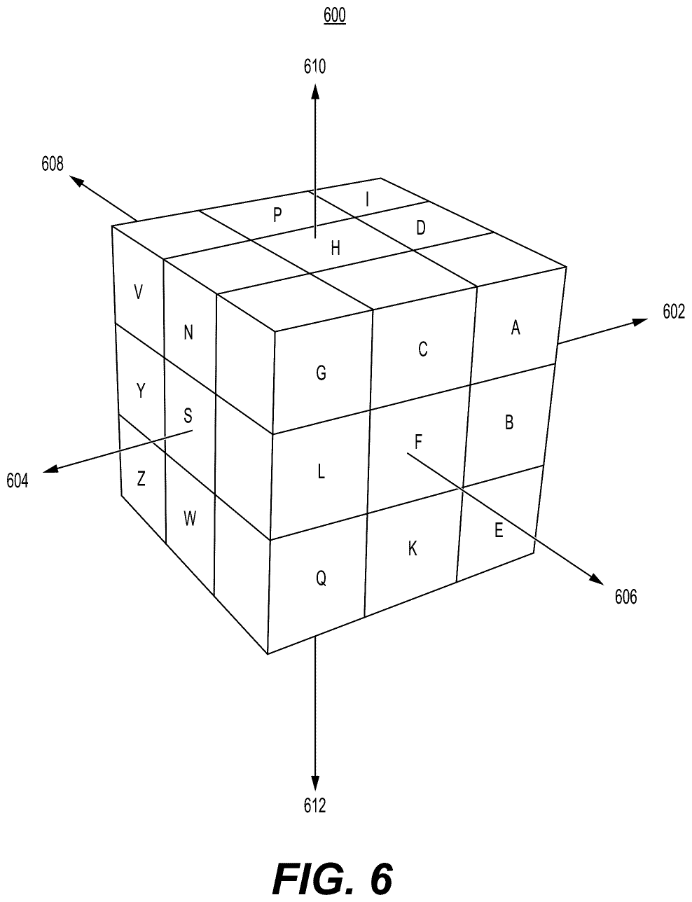

Chart 520 is a single dimension delta comparing the lateral extension gap 116 to the medial extension gap 114. Similar charts may be made comparing the medial flexion gap 118 to the medial extension gap 114, and the lateral extension gap 120 to the medial extension gap 114, resulting in a set of three deltas: medial extension delta; lateral flexion delta; and medial flexion delta. The examples described in FIGS. 6-8 relate to the comparison of other gaps to a base medial extension gap 114. However, any of the four gaps may be used as a base with the three deltas being based on that base gap. For example, if the lateral extension gap 116 was used as a base instead of the medial extension gap 114, there would still be three deltas conveying the same information: the lateral extension delta; lateral flexion delta; and medial flexion delta. FIGS. 6-8 will now be described with reference to an example wherein the deltas are measured relative to the medial extension gap.

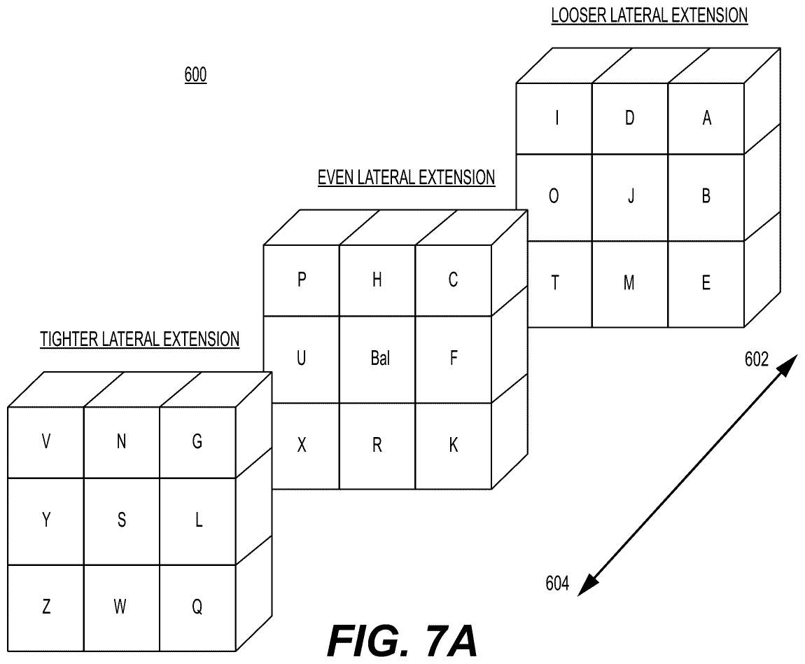

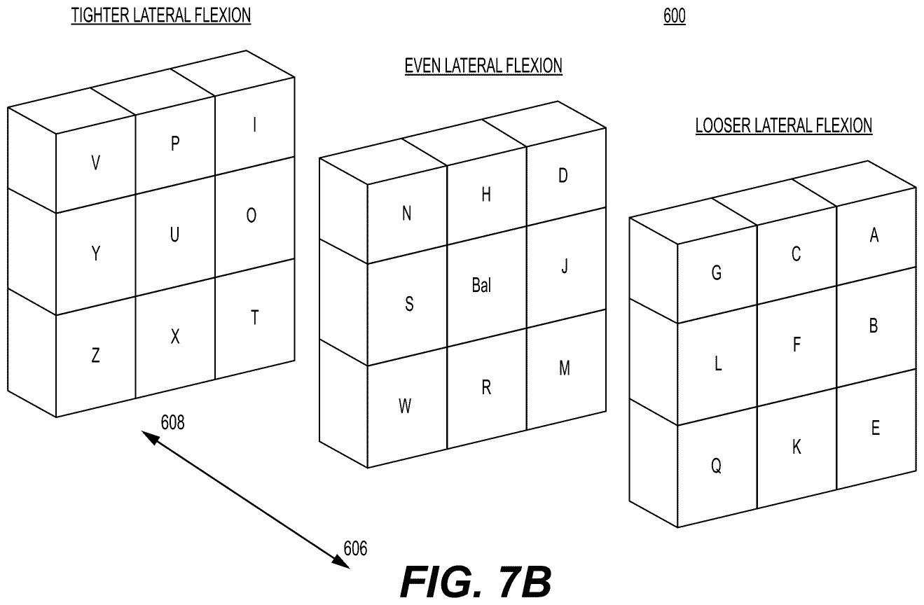

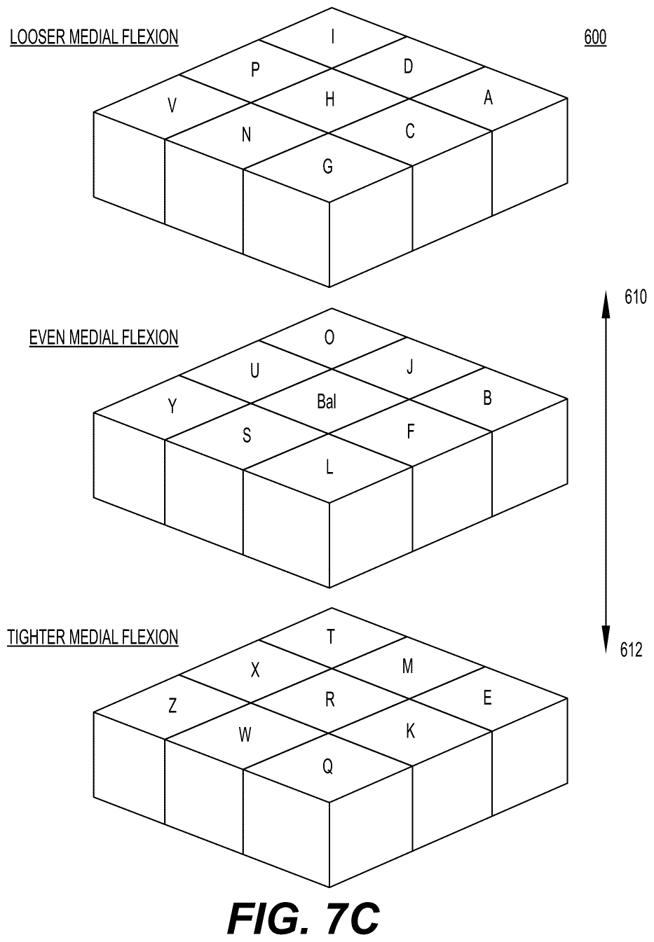

FIG. 6 illustrates a 3-dimension depiction of ligament laxity data. The 3-dimensional depiction may take the form of a classification cube 600 for providing a user, such as a surgeon, nurse, or other professional, a visual aid for a soft tissue balance assessment during the preoperative or intraoperative stages of a knee replacement procedure. The classification cube 600 may be structured like a Rubik's cube including 27 smaller cubes, labeled with the letters A to Z (26 cubes) and a central cube labeled “Bal” for “balanced.” (See FIGS. 7A-7C). Each smaller cube includes three classifications or coordinates, one for each of the three deltas: a first gap delta, a second gap delta, and a third gap delta. The first gap delta is shown along a first axis extending through directions 602 and 604, with first direction 602 being a looser gap than the base gap and direction 604 being a tighter gap than the base gap. In the example embodiment, this translates to direction 602 representing a looser first gap delta. Conversely, direction 604 represents a tighter first gap delta. Similarly, direction 606 represents a looser second gap delta, direction 608 represents a tighter second gap delta, direction 610 represents a looser third gap delta, and direction 612 represents a tighter third gap delta.

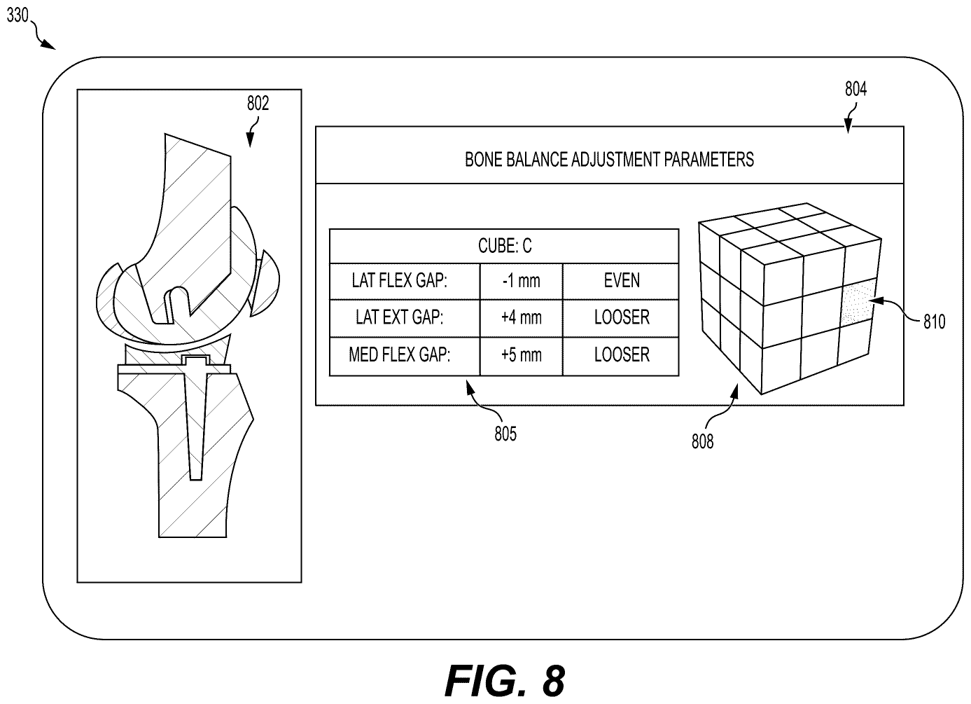

In the example embodiment shown in FIGS. 6-8, the medial extension is the base quadrant used to measure the gap deltas. As such, the first gap delta may be the lateral extension delta, the second gap delta may be the lateral flexion delta, and the third gap delta may be the medial flexion delta. Any of the gaps may be designated the base quadrant, and any of the three gap deltas based on the base quadrant may be designated first, second, or third gap deltas. The interface shown in FIG. 8 may be customizable to allow for a user to set the base quadrant and set the preferred gap deltas, and may also include a default base quadrant and first through third gap deltas.

The three gap delta coordinates may be ascertained from the position of each smaller cube within the larger cube. FIGS. 7A-7C illustrate exploded views of the classification cube 600 in the three directions. FIG. 7A is exploded in the first direction, along axis 602-604, which indicates the value of the first gap delta for each of the cubes. FIG. 7B is exploded in the second direction, along axis 606-608, which indicates the value of the second gap delta for each of the cubes. FIG. 7C is exploded in the third direction, along axis 610-612, which indicates the value of the third gap delta for each of the cubes.

In the example embodiment, the first gap delta is a lateral extension delta, which is defined by the difference between the lateral extension gap and the medial extension gap, as the medial extension is the base quadrant in the example used. The cubes in the rank in the direction of 602 are all of the cubes that indicate a looser lateral extension gap. The cubes in the rank in the direction of 604 are all of the cubes that indicate a tighter lateral extension gap. The cubes in the middle rank are all of the cubes that indicate an even lateral extension gap. The demarcation between looser and even, and even and tighter, may be determined based on empirical data and may be adjusted with updated data or information. The adjustment may me made automatically by balancing system 300 and/or manually by a practitioner such as a surgeon or administrator. Table 1 below indicates the first gap delta value for each of the cubes shown in FIG. 7A.

TABLE 1

| Cube | First Delta |
|---|---|
| Bal | Even |
| A | Looser |
| B | Looser |
| C | Even |
| D | Looser |
| E | Looser |
| F | Even |
| G | Tighter |
| H | Even |
| I | Looser |
| J | Looser |
| K | Even |
| L | Tighter |
| M | Looser |
| N | Tighter |
| O | Looser |
| P | Even |
| Q | Tighter |
| R | Even |
| S | Tighter |
| T | Looser |
| U | Even |
| V | Tighter |
| W | Tighter |
| X | Even |
| Y | Tighter |
| Z | Tighter |

With reference to FIG. 7B, the second gap delta is indicated by the ranks in the direction of the axis 606-608. The cubes in the rank in the direction of 606 are all of the cubes that indicate a looser lateral flexion gap. The cubes in the rank in the direction of 608 are all of the cubes that indicate a tighter lateral flexion gap. The cubes in the middle rank are all of the cubes that indicate an even lateral flexion gap. Table 2 below indicates the second gap delta value for each of the cubes shown in FIG. 7B.

TABLE 2

| Cube | Second Delta |
|---|---|
| Bal | Even |
| A | Looser |
| B | Looser |
| C | Looser |
| D | Even |
| E | Looser |
| F | Looser |
| G | Looser |
| H | Even |
| I | Tighter |
| J | Even |
| K | Looser |
| L | Looser |
| M | Even |
| N | Even |
| O | Tighter |
| P | Tighter |
| Q | Looser |
| R | Even |
| S | Even |
| T | Tighter |
| U | Tighter |
| V | Tighter |
| W | Even |
| X | Tighter |
| Y | Tighter |
| Z | Tighter |

With reference to FIG. 7C, the third gap delta is indicated by the ranks in the direction of the axis 610-612. The cubes in the rank in the direction of 610 are all of the cubes that indicate a looser medial flexion gap. The cubes in the rank in the direction of 612 are all of the cubes that indicate a tighter medial flexion gap. The cubes in the middle rank are all of the cubes that indicate an even medial flexion gap. Table 3 below indicates the third gap delta value for each of the cubes shown in FIG. 7C.

TABLE 3

| Cube | Third Delta |
|---|---|
| Bal | Even |
| A | Looser |
| B | Even |
| C | Looser |
| D | Looser |
| E | Tighter |
| F | Even |
| G | Looser |
| H | Looser |
| I | Looser |
| J | Even |
| K | Tighter |
| L | Even |
| M | Tighter |
| N | Looser |
| O | Even |
| P | Looser |
| Q | Tighter |
| R | Tighter |
| S | Even |
| T | Tighter |
| U | Even |
| V | Looser |
| W | Tighter |
| X | Tighter |
| Y | Even |
| Z | Tighter |

Table 4 provides the information from tables 1-3 compiled into a single table. Each smaller cube indicates a unique set of three parameters indicating the first delta, the second delta, and the third delta, with each delta being indicated as “looser,” “tighter,” or “even.”

TABLE 4

| Cube | First Delta | Second Delta | Third Delta |
|---|---|---|---|
| Bal | Even | Even | Even |
| A | Looser | Looser | Looser |
| B | Looser | Looser | Even |
| C | Even | Looser | Looser |
| D | Looser | Even | Looser |
| E | Looser | Looser | Tighter |
| F | Even | Looser | Even |
| G | Tighter | Looser | Looser |
| H | Even | Even | Looser |
| I | Looser | Tighter | Looser |
| J | Looser | Even | Even |
| K | Even | Looser | Tighter |
| L | Tighter | Looser | Even |
| M | Looser | Even | Tighter |
| N | Tighter | Even | Looser |
| O | Looser | Tighter | Even |
| P | Even | Tighter | Looser |
| Q | Tighter | Looser | Tighter |
| R | Even | Even | Tighter |
| S | Tighter | Even | Even |
| T | Looser | Tighter | Tighter |
| U | Even | Tighter | Even |
| V | Tighter | Tighter | Looser |
| W | Tighter | Even | Tighter |
| X | Even | Tighter | Tighter |
| Y | Tighter | Tighter | Even |
| Z | Tighter | Tighter | Tighter |

FIG. 8 illustrates an exemplary graphical user interface of the bone balancing system 300 displayed on the display 330. Referring to FIGS. 6-8, a practitioner (e.g., surgeon) may use a computer or other system of the bone balancing system 300 to analyze one or more images of a patient's anatomy 802 (e.g., knee joint) and/or an initially determined procedure plan 320. The one or more images of the patient's anatomy 802 and/or the procedure plan 320 may be included in a case file, patient folder, etc. that is selectable by the practitioner. The one or more images of the patient's anatomy 802 may have been acquired using an image acquisition device 310 and/or with the patient in various poses. The bone balancing system 300 may analyze the one or more images of the patient's anatomy 802 to detect and/or scan for the ligament laxity information in the form of flexion and extension gaps. The bone balancing system 300 may cause to be displayed on display 330 the information regarding the ligament laxity information in user-readable form 806. This information may include values for the four gaps 114-120, values for the three gap deltas, as shown in FIG. 8, or a combination of both. Furthermore, the display may also include an indication of the cube corresponding to the ligament laxity information. The display may also include a graphical indication of the cube 600 with the smaller cube corresponding to the patient's anatomy 802 highlighted. The interface may also be configured to allow for a color coded visualization of the ligament laxity information, e.g., green for balanced, red for all deltas tighter, yellow for all deltas looser, and blue for a combination, etc.

The interface may also be updated in real time from the image analysis system 300 both preoperatively and intraoperatively. As the course of treatment is continued, the actual or observed outcomes and/or results 350 may also be used by the bone balancing system 300 to either update its predictions (e.g., intraoperatively based on intraoperatively collected data or outcomes) and/or to make future predictions for future patients (e.g., based on a postoperative result or outcome). Intraoperative data for further refinements may be similar to the preoperative data 420, and the ligament laxity information including the values for the four gaps 114-120 and the corresponding gap deltas may be updated based on the sensed position of the knee via image analysis system 300. In some examples, this may include a change in the colors of the cubes as the surgeon performs an operation, and changes in which cube is indicated graphically on the display 330.

In the example shown in FIG. 8, the bone balancing system 300 may have analyzed the one or more images of the patient's anatomy 802 to have found a medial extension gap of 19 mm, a lateral flexion gap of 18 mm, a lateral extension gap of 23 mm, and a medial flexion gap of 24 mm. Correspondingly, using the medial extension gap as a base, the lateral extension gap delta is determined to be −1 mm, the lateral flexion gap delta is determined to be +4 mm, and the medial flexion gap is determined to be +5 mm. Because a lateral extension gap delta between −2 mm and +2 mm is determined to be “even,” the lateral extension gap delta of −1 mm falls within the “even” subset. Similarly, because gap deltas greater than 2 mm are determined to be “looser,” the lateral flexion gap delta of +4 mm and the medial flexion gap delta of +5 mm are indicated as looser. As indicated above, the demarcations between “tighter,” “even,” and “looser” may be based on empirical data from a historical patient set, and may be adjusted based on updated data or at a user's discretion. As shown in Table 4, a patient with these gap deltas is represented by cube C for even first delta, and looser second and third deltas.

Aspects disclosed herein may provide one or more algorithms to determine one or more bone resection parameters and/or implant parameters and predict soft tissue effects, allowing a surgeon to automate preoperative and/or intraoperative decision making. The algorithm may help streamline a workflow for a procedure to anticipate soft tissue balancing, and to proactively adjust bone cuts accordingly.

Aspects disclosed herein may be used to sense or collect preoperative, intraoperative, and/or postoperative information about a patient and/or a procedure.

Aspects disclosed herein contemplate implants or prosthetics, and are not limited to the contexts described. For example, implants disclosed herein may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the implants or implant systems may include sensors such as inertial measurement units, strain gauges, accelerometers, ultrasonic or acoustic sensors, etc. configured to measure position, speed, acceleration, orientation, range of motion, and/or sensors configured to measure biometric changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. The implants may, for example, be a sensor or other measurement device configured to be drilled into a bone, another implant, or otherwise implanted in the patient's body.

Aspects and systems disclosed herein may make determinations based on images or imaging data (e.g., from CT scans). Images disclosed herein may display or represent bones, tissues, or other anatomy, and systems and aspects disclosed herein may recognize, identify, classify, and/or determine portions of anatomy such as bones, cartilage, tissue, and bone landmarks, such as each specific vertebra in a spine. Aspects and systems disclosed herein may determine relative positions, orientations, and/or angles between recognize bones, such as a Cobb angle, an angle between a tibia and a femur, and/or other alignment data.

Aspects and systems disclosed herein provide displays having graphical user interfaces configured to graphically display data, determinations, and/or steps, targets, instructions, or other parameters of a procedure, including preoperatively, intraoperatively, and/or postoperatively. Figures, illustrations, animations, and/or videos displayed via user interfaces may be recorded and stored on the memory system.

Aspects and systems disclosed herein may be implemented using machine learning technology. One or more algorithms may be configured to learn or be trained on patterns and/or other relationships across a plurality of patients in combination with preoperative information and outputs, intraoperative information and outputs, and postoperative information and outputs. The learned patterns and/or relationships may refine determinations made by one or more algorithms and/or also refine how the one or more algorithms are executed, configured, designed, or compiled. The refinement and/or updating of the one or more algorithms may further refine displays and/or graphical user interfaces (e.g., bone recognition and/or determinations, targets, recognition and/or display of other conditions and/or bone offsets, etc.).

Aspects disclosed herein may be configured to optimize a “fit” or “tightness” of an implant provided to a patient during a medical procedure based on detections by the one or more algorithms. A fit of the implant may be made tighter by aligning the implant with a shallower bone slope and/or determining a shallower resulting or desired bone slope, by increasing a thickness or other dimensions of the implant, by determining certain types of materials or a type of implants or prosthesis (e.g., a stabilizing implant, a VVC implant, an ADM implant, or an MDM implant). A thickness of the implant may be achieved by increasing (or decrease) a size or shape of the implant. Tightness may be impacted by gaps and/or joint space width, which may be regulated by an insert which may vary depending on a type of implant or due to a motion. Gaps may be impacted by femoral and tibial cuts. Tightness may further be impacted by slope. A range of slope may be based on implant choice as well as surgical approach and patient anatomy. A thickness of the implant may also be achieved by adding or removing an augment or shim. For example, augments or shims may be stackable and removable, and a thickness may be increased by adding one or more augments or shims or adding an augment or shim having a predetermined (e.g., above a certain threshold) thickness. Fit or tightness may also be achieved with certain types of bone cuts, bone preparations, or tissue cuts that reduce a number of cuts made and/or an invasiveness during surgery.

Aspects disclosed herein may be implemented during a robotic medical procedure using a robotic device. Aspects disclosed herein are not limited to specific scores, thresholds, etc. that are described. For example, outputs and/or scores disclosed herein may include other types of scores such as the hip disability and osteoarthritis score or HOOS, KOOS, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes, implant designs, and/or preoperative analyses, predictions, or workflows. Aspects disclosed herein may augment the continuum of care to optimize post-operative outcomes for a patient. Aspects disclosed herein may recognize or determine previously unknown relationships, to help optimize care, predict soft tissue laxity, and/or to optimize design of a prosthetic or implant.

What is claimed is:

1. A computer-implemented method for generating and presenting a display of a visualization of joint parameters, the method comprising:
receiving, by one or more processors, imaging data of a joint;
extracting, by the one or more processors, gap parameter information from the imaging data,
calculating, based on the gap parameter information, delta parameter information;
generating, by the one or more processors, a graphical user interface (GUI) based on the delta parameter information, wherein the GUI includes a three- dimensional element configured to represent the delta parameter information;
displaying, by the one or more processors, the generated GUI on an electronic display.

2. The computer-implemented method of claim 1, wherein the gap parameter information includes a first gap parameter value, a second gap parameter value, a third gap parameter value, and a fourth gap parameter value.

3. The computer-implemented method of claim 1, wherein the delta parameter information includes a first delta parameter value, a second delta parameter value, and a third delta parameter value.

4. The computer-implemented method of claim 1, wherein the three-dimensional element comprises a plurality of sub-elements, each of the plurality of sub-elements corresponding to a pre-determined first delta parameter value, a pre-determined second delta parameter value, and a pre-determined third delta parameter value.

5. The computer-implemented method of claim 2, wherein
calculating the delta parameter information comprises:
calculating a first difference between the first gap parameter value and the second gap parameter value;
calculating a second difference between the first gap parameter value and the third gap parameter value; and
calculating a third difference between the first gap parameter value and the fourth gap parameter value.

6. The computer-implemented method of claim 5, wherein
calculating the delta parameter information further comprises:
determining the first delta parameter value based on the first difference;
determining the second delta parameter value based on the second difference; and
determining the third delta parameter value based on the third difference.

7. The computer-implemented method of claim 2, wherein:
the first gap parameter value is a measure of a medial extension gap in a knee joint of a patient;
the second gap parameter value is a measure of a lateral extension gap in the knee joint of the patient;
the third gap parameter value is a measure of a medial flexion gap in the knee joint of the patient; and
the fourth gap parameter value is a measure of a lateral flexion gap in the knee joint of the patient.

8. The computer-implemented method of claim 1, wherein the three-dimensional element is a cube with twenty-seven sub-elements, wherein each sub-element is a cube.

9. The computer-implemented method of claim 8, wherein a central sub-element of the three dimensional element is configured to represent gap parameter information including:
a lateral extension gap of the joint within +/−2 mm of a medial extension gap of the joint;
a medial flexion gap of the joint within +/−2 mm of the medial extension gap of the joint, and
a lateral flexion gap of the joint all within +/−2 mm of the medial extension gap of the joint.

10. A system for generating and presenting a display of a visualization of joint parameters, comprising:
a computer-readable storage medium storing instructions for generating and presenting a display of guidance for performing the robotic medical procedure; and
one or more processors configured to execute the instructions to perform a method including:
receiving imaging data of a joint;
determining gap parameter information based on the imaging data,
generating a graphical user interface (GUI) configured to display a three-dimensional element comprising a plurality of sub-elements;
determining which of the plurality of sub-elements correspond to the determined gap parameter information; and
generating a display of the GUI, wherein at least one sub-element is associated with the determined gap parameter information.

11. The system of claim 10, wherein each of the plurality of sub-elements corresponds to a pre-determined first delta parameter value, a pre-determined second delta parameter value, and a pre-determined third delta parameter value.

12. The system of claim 11, wherein:
the first delta parameter value is based on a first difference between a first gap parameter value and a second gap parameter value;
the second delta parameter value is based on a second difference between the first gap parameter value and a third gap parameter value; and
the third delta parameter value is based on a third difference between the first gap parameter value and a fourth gap parameter value.

13. The system of claim 12, wherein:
the first gap parameter value is a measure of a medial extension gap of the joint;
the second gap parameter value is a measure of a lateral extension gap of the joint;
the third gap parameter value is a measure of a medial flexion gap of the joint; and
the fourth gap parameter value is a measure of a lateral flexion gap of the joint, wherein the joint is a knee joint.

14. The system of claim 12, wherein:
the first gap parameter value is a measure of a medial flexion gap of the joint;
the second gap parameter value is a measure of a lateral flexion gap of the joint;
the third gap parameter value is a measure of a medial extension gap of the joint; and
the fourth gap parameter value is a measure of a lateral extension gap of the joint, wherein the joint is a knee joint.

14. The system of claim 10, wherein the three-dimensional element is a cube with twenty-seven sub-elements.

15. The system of claim 14, wherein twenty-six of the twenty-seven sub-elements is represented by a letter of the alphabet, and displaying the determined sub-element includes displaying the letter of the alphabet representing the determined sub-element.

16. A computer-implemented method for generating a display for visualization of joint parameters, the method comprising:
receiving, by one or more processors, imaging data of a joint;
extracting, by the one or more processors, gap parameter information from the imaging data;
calculating, by the one or more processors, based on the gap parameter information, delta parameter information;
displaying, by the one or more processors, the delta parameter information on a graphical user interface (GUI).

17. The computer-implemented method of claim 16, wherein:
the gap parameter information includes a first gap parameter value, a second gap parameter value, a third gap parameter value, and a fourth gap parameter value; and
the delta parameter information includes a first delta parameter value, a second delta parameter value, and a third delta parameter value.

18. The computer-implemented method of claim 17, wherein:
the first delta parameter value is based on a first difference between the first parameter value and a second parameter value;
the second delta parameter value is based on a second difference between the first parameter value and a third parameter value; and
the third delta parameter value is based on a third difference between the first parameter value and a fourth parameter value.

19. The computer-implemented method of claim 18, wherein:
the first gap parameter value is a measure of a medial extension gap of the joint;
the second gap parameter value is a measure of a lateral extension gap of the joint;
the third gap parameter value is a measure of a medial flexion gap of the joint; and
the fourth gap parameter value is a measure of a lateral flexion gap of the joint, wherein the joint is a knee joint.

20. The computer-implemented method of claim 16, wherein the three-dimensional element is a cube with twenty-seven sub-elements.
